(19) United States
(12) Patent Application Publication
(10) Pub. No.: US 2026/0363998 A1
(43) Pub. Date: Nov. 19, 2026

(54) LONG WEAR LIP COLOR FORMULATION

(71) Applicant: Coty Inc., New York, NY (US)

(72) Inventors: Camille Le Jolu, New York, NY (US); Tatyana Zamyatin, New York, NY (US); Debra J. Coleman Nally, New York, NY (US); Sepideh Karkouti, New York, NY (US)

(21) Appl. No.: 19/135,720

(22) PCT Filed: Dec. 13, 2023
(86) PCT No.: PCT/US2023/083926

§ 371 Date: Jun. 4, 2025

Related U.S. Application Data
(60) Provisional application No. 63/387,414, filed on Dec. 14, 2022.

(51) Int. Cl. A61K 8/891; A61K 8/31; A61K 8/92; A61Q 1/06

(52) U.S. Cl. CPC A61K 8/891; A61K 8/31; A61K 8/922; A61Q 1/06; A61K 2800/43

(57) ABSTRACT

Various examples disclosed relate to long wear liquid lip color. The present disclosure includes formulations and methods of making a lip color with true long wear and low transferability. The formulations can include a resin containing base coat composition insoluble with a top coat composition. The base coat composition can include adhesive film-forming silicone-based resins, at least one pigment, and a plasticizer, dispersed in a solution. The top coat composition insoluble with the base coat composition can be dispersed within the base coat composition.

### CLAIM OF PRIORITY

This application claims the benefit of priority to U.S. Provisional Application Ser. No. 63/387,414, filed on Dec. 14, 2022, which is incorporated herein by reference in its entirety.

### TECHNICAL FIELD

The present subject matter relates to cosmetics and, more particularly, to lip products and lip color. More specifically, the present disclosure relates to a long-wear liquid lipstick or long-wear lip gloss formulations.

### BACKGROUND

Lip color has been popular as a cosmetic for hundreds of years. Such lip color can be applied to produce a desired color or texture thereon. Lip color products can optionally protect the lips from sunburn, chapping, or other effects of the environment or weather. In some cases, lip color products can include wax, oils, emollients, or other components that can provide structure to the lip color. Other components, such as oils, emollients, pigments, or others, can be included to provide color, texture, and benefits for lip product application.

In recent decades, lipstick, lip gloss, lip stains, lip balms, lip liners, and liquid lipstick, among other products, have been popular amongst cosmetics users. Often, a consumer may desire to apply a lip color for a particular period of time, wishing the color and texture of the product to last for a suitable amount of time. Many types of lip color products can fade over time when worn. Long-wear lip color, which truly lasts for an extended period of time, can be rare. Additionally, feel and comfort of the lip color product can be important. Some materials can, for example, cause a user's lip to feel dry over an extended period of time. It is desirable therefore to produce materials that are comfortable to the user and include long lasting color.

### SUMMARY OF THE DISCLOSURE

In a first example, a long-wear lip color formulation includes a base coat composition having a cyclic (e.g., cyclopentasiloxane or “D5”) content of less than 0.1% by weight and a top coat composition. The bae coat includes a first resin comprising polyphenylsilsesquioxane and a second resin comprising trimethylsiloxysilicate, a plasticizer comprising dimethicone, a thickener, at least one pigment, and a solvent. The first resin, the second resin, the thickener, the plasticizer, and the at least one pigment are in the solvent. The ratio of trimethylsiloxysilicate to polyphenylsilsesquioxane is about 4:1 to about 7:1 in the base coat composition. Once the base coat composition and the top coat composition are applied sequentially, the long-wear lip color formulation having a wear time of about 24 hours or more.

In a second example, a long-wear lip color composition includes a base coat composition and a top coat composition. The base coat composition comprises base coat components including a film-forming silicone-based resin, at least one pigment, a thickener, and a plasticizer, in a solution. The silicone-based resins include at least an MQ resin and an MT resin, having a ratio of about 3:1 to about 8:1. The top coat composition is applied sequentially on the base coat composition to create a long-wearing film on an integument. The long-wearing film on an integument has a wear time of at least 24 hours.

In a third example, a formulation includes a trimethylsiloxysilicate and polyphenylsilsesquioxane resin-based long wear lip color. The formulation is made by a process of premixing the trimethylsiloxysilicate resin with a solvent and adding a plasticizer to make a preliminary mixture, heating the preliminary mixture above the glass transition temperature for polypheylsilsesquioxane to produce a heated mixture, suspending the polyphenylsilsesquioxane resin in the heated mixture and softening the heated mixture to produce a combined mixture, letting the combined mixture stand for a predetermined time to thicken and stabilize to produce an base coat composition, and combining the base coat composition with a thickener and optionally one or more pigments to produce the formulation. The lip color has a wear time of about 24 hours or more.

### DETAILED DESCRIPTION

Reference will now be made in detail to certain examples of the disclosed subject matter. While the disclosed subject matter will be described in conjunction with the enumerated claims, it will be understood that the exemplified subject matter is not intended to limit the claims to the disclosed subject matter.

The present disclosure provides formulations and methods for long-wear lip color, lasting over 24 hours. The discussed long wear lip color formulations allow for improved product aesthetics and feel due to a film-forming resin system. The long wear lip color technology discussed here can delight consumers with noticeable, superior, long-wearing appearance, including color and finish, while delivering a parity feel and lip condition compared to conventional lip colors. The formulations discussed herein can be used for a variety of lip color products, such as in liquid, semi-solid, or solid products. A variety of aesthetics, such as varying feels, finishes, glosses, flavors, and fragrances, can also be used.

Typically, current lip color products are conventional lipstick bullets or dip-in liquids having a good initial appearance, color, finish, and feel, but limited staying power. That is, these types of lip color can easily transfer off lips, or be removed, such as by eating and drinking. Some “long-wear” lip color products have been introduced, but these products typically have a mediocre staying power, coming off after only a few hours, or after eating or drinking. These colors can also appear overtly dry during wear. Lip color “kits” have been introduced which may include a base coat and a clear gloss top coat. However, these colors have poor finish and feel performance compared to conventional lipstick bullets or dip-in liquids. Moreover, these kits often include a high number of volatile hydrocarbons and silicones that cause a loss of hydration and overall lip dryness over time. These formulations are generally not green and contain a higher amount of cyclic content.

By contrast, the lip color formulations discussed herein have long-wearing color and finish, a formulation that has good feel to the user, and are environmentally friendly, such as by having a low cyclopentasiloxane (D5) (“cyclic”) content. The lip color technology provides long-wearing appearance for both color and finish. The composition itself includes a base coat composition and a top coat composition that are applied sequentially.

The base coat composition includes film-forming silicone-based resins and pigments in solution, in addition to a clean plasticizer content. Additional elements can be included in the base coat composition, such as pigments, thickening or structural agents, wetting or dispersing agents, plasticizers or tackifiers, or other components.

### DEFINITIONS

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “solvent” as used herein refers to a liquid that can dissolve a solid, liquid, or gas. Non-limiting examples of solvents are silicones, organic compounds, water, alcohols, ionic liquids, and supercritical fluids.

As used herein, the term “polymer” or “resin” refers to a molecule having at least one repeating unit and can include co-resins.

The resins described herein can terminate in any suitable way. In some examples, the resins can terminate with an end group that is independently chosen from a suitable resinization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

The term “stick” as used herein, refers to cosmetic compositions molded into the form of a stick. For some embodiments, the compositions are heated until molten and then poured into a mold and cooled. Stick embodiments also include anhydrous compositions capable of being formed into sticks.

As used herein, the term “cyclic” or “cyclics” refer to cyclic silicone, such as cyclopentasiloxane (D5), content in formulations. Cyclics can be non-volatile and are considered not “green” or environmentally friendly in cosmetics formulations.

Embodiments of the invention are described in sufficient detail to enable those skilled in the art to practice the invention. Other embodiments may be utilized, and structural, logical, chemical, and other changes may be made without departing from the spirit or scope of the invention discussed herein. The following detailed description is, therefore, not to be taken in a limiting sense, and the scope of the invention discussed herein is defined only by the appended claims.

### Lip Color Composition

Various formulation herein include a resin-based long wear lip color having a base coat having a base coat composition and top coat composition for sequential application. The lip color formulation is environmentally friendly, containing low cyclic content. The base coat composition includes silicone-based resins, at least one pigment, and a plasticizer, in a solution. The silicone-based resins include at least an Me₃SiO unit and SiO₄ unit silicone (e.g., M unit and Q unit or “MQ”) resin and an Me₃SiO unit and MeSiO₃ unit silicone (e.g., M unit and T unit or “MT”) resin, with an MT:MQ ratio of about 3:1 to about 7:1. The at least one pigment can optionally be treated. The base coat can also include a gellant or thickener. The top coat composition include components that are insoluble with the base coat to add gloss and feel without removing the base coat composition. Specifically, the top coat composition includes a gellant or thickener and top coat formulation. In some cases, various components can be in both or different base and top coat compositions.

The lip color is substantially transfer proof and long wearing. To achieve the benefits of long-wear and good feel, the lip color includes two insoluble compositions: the base coat composition and the top coat composition, for sequential application. Each of the compositions provides distinct benefits that are complementary to the total composition performance. Upon application to the skin or an integument, the base coat composition delivers long-wearing color benefits by providing transfer-resistance of pigments and adhesion to lips or another integument. The base coat composition still allows for good feel in addition to long wear and is formulated with low cyclic content. Applied after the base coat composition, the top coat composition forms a barrier layer over the surface of the base coat composition. The top coat composition delivers gloss or shine, and lip feel, such as moisture, or other benefits to the surface of the pigmented base coat composition layer.

In an example formulation, the base coat composition includes a first resin, a second resin, and a plasticizer. The first resin can be, for example, a trimethylsiloxysilicate suspended in a solvent. The second resin can be a polyphenylsilsesquioxane. The plasticizer can be dimethicone 1Mcst with low cyclic content (D4/D5/D6<0.1%). The formulation can additionally include a thickener, such as a hectorite clay, disteardimonium hectorite, or a propylene carbonate gellant. Additional components, such as coated pigments, and other components, can be included in the base coat composition and/or the top coat composition.

Base Coat Composition. The base coat composition includes at least a first resin, a second resin, a plasticizer, a thickener, and at least one pigment, all in a solvent. The based coat composition has a viscosity of about 3,500 cPs to about 110,000 cPs. In an example, the base coat composition has a minimum viscosity of about 5,000 cPs, of about 7,500 cPs, of about 10,000 cPs, or of about 15,000 cPs.

The base coat components are dispersed and/or suspended in a solvent. In an example, the solvent is isododecane. In an example, the first resin (e.g., MQ resin) is suspended in the solvent. In an example, the second resin (e.g., MT resin), in addition to the plasticizer, the thickener, and the pigment are dispersed in the solvent.

The base coat composition, when applied sequentially with the top coat composition, has a wear time of at least 24 hours. The long-wear lip color formulation has a wear time of about 24 hours or more according to consumer clinical screening.

Resins. The base coat composition includes at least one flexible, adhesive film-forming resin which is in solvent. The one or more resins can form a flexible, skin-adhesive film that is resistant to removal such as by blotting or rubbing once applied to the lips. Film-forming resins can allow for more favorable health conditions and overall feel. Such film-forming resins can form colloidal dispersions of film forming resin particles electrostatically or sterically stabilized in solvent. These resins can form films through a process of particle coalescence. In an example, the solvent in which the resin is dispersed or suspended evaporates or absorbs into a substrate, until the resin particles reach a close packing. Stabilizing forces around each particle are overcome by a combination of interfacial tension and capillary forces as the solvent continues to evaporate, forming particle contact and deformation. Here, a foam-like packing structure develops. The interface between the particles gradually disappears as interdiffusion of resin molecules between neighboring particles occurs, forming a continuous layer or film. A variety of specific resins can lend themselves to such film-forming tendencies. Such resins can be formed, for example, by chain-growth polymerization or step-growth polymerization processes.

In an example, the resins used in the lip color formulation herein can include at least one MQ and one MT resin, such as at an MT:MQ ratio of about 3:1 to about 8:1, of about 4:1 to about 7:1, or of about 5:1. MT resins are typically more flexible than MQ resins, allowing for a better overall consistency of the lip color without additional plasticizer.

The first resin (e.g., the MQ resin) can be about 3% to about 10% by weight of the base coat composition. The second resin (e.g., the MT resin) can be about 10% to about 60% by weight of the base coat composition.

For example, polyphenylsilsesquioxane and trimethylsiloxysilicate can be used. The use of an MT resin such as polyphenylsilsesquioxane allows for an increase in the resin content without increasing plasticizer need.

Solids content of resin in all of these film-forming versions can range from about 10% up to about 75% by weight of the resin dispersion, or about 10% to about 50% of the resin dispersion.

The resins can have beneficial dry film mechanical properties to help achieve a preferred lip color feel and long wear properties. For example, the achieve desired film adhesion and wear on skin, while minimizing adverse feel effects such as tackiness, stiffness, or tightness, the dried resin film can have particular storage modulus, loss modulus, and tan delta. For example, in a dry form the resin film can have a storage modulus (G′) of about 1×10⁴ Pa to about 5×10⁶ Pa. In another example, in a dry form the resin film can have a loss modulus (G″) of about 3×10³ Pa to about 5×10⁶ Pa. In another example, in a dry form the resin film can have tan δ (G′/G′) of about 0.30 to about 1.0. or about 0.50 to about 1.0.

Plasticizers and Tackifiers. In addition to the film-forming resins, the base coat composition includes at least one plasticizer and/or tackifier to impact one or more aspects of the film formation and adhesion to skin. These agents are used to affect drying or setting rates of the resins, and influence final film properties such as flexibility, adhesion, and cohesion.

The plasticizer chosen has a low cyclic content of about 0.1% or less by weight. This helps keep the formulation green while providing flexibility to the formulation.

In an example formulation, dimethicone is used as the plasticizer. Specifically, dimethicone Mcst is used. In this case, the dimethicone has a low cyclic count such a D4/D5/D6 or less than about 0.1%.

The plasticizer can have a viscosity of at least about 900,000 cPs. The plasticizer can be about 20% or less by weight of the composition. For example, a dimethicone plasticizer used at about 12% to about 16% by weight provides flexibility, and good tack profiles for the lip color formulation. In an example, the plasticizer can have a minimum viscosity of about 900,000 cPs, or a viscosity range of about 900,000 cPs to about 1,000,000 cPs.

Such a plasticizer is incorporated directly into the base coat composition with the film-forming resins and pigments. In some cases, the plasticizer can be additionally or alternatively incorporated into the top coat composition. The amount of resins and plasticizer in the lip formulation can affect the overall flexibility and tack in the formulation.

Pigments and Colorants. In addition to the film-forming resins and plasticizers, the base coat composition includes at least one colorant or pigment. A wide range of cosmetically acceptable colorants can be used to achieve a range of colors and effects typical for lip color. In an example, the pigment is a treated pigment, such as a triethoxycaprylylsilane (TC) treated pigment.

For example, inorganics such as iron oxides (e.g., red, yellow, black) and titanium dioxide can be used. In an example, organics such as the FD&C and D&C dyes and lakes can be used. Additionally, micas and pearlescent powders can be used, such as to achieve additional color and optical effects such as luster or sparkle. In some cases, other complementary colorants, such as pigments, dyes, mica or other pearlescent agents, can also be added to the separating top coat composition to impart additional color, luster, and/or highlight effects to the applied composition.

Wetting and Dispersing Agents. In addition to the film-forming resins, the base coat composition can include wetting or dispersing agents to facilitate adhesion and wetting of the lip color composition over the applied surface area, and to facilitate wetting and dispersion of colorants added to the base coat composition. In some cases, wetting or dispersing agents can affect a strong reduction in the surface tension of a solvent used in the base coat composition. This can improve substrate wetting and leveling of the lip color on lower energy hydrophobic surfaces such as pigments and skin. This can allow for enhanced adhesion on skin.

Example wetting and dispersing agents can include polyether-modified polydimethylsiloxanes, such as dimethicone copolyols and polyether-modified polydimethylsiloxanes.

Thickeners and Structing Agents. In addition to the film-forming resins, the base coat composition includes a thickener and/or structuring agent to increase the viscosity and structure of the pigmented resin layer. This prevents settling or sedimentation of pigment particles in the lip color over time. Such thickeners and/or structuring agents can also help control application and spreading of the lip color across lips by providing even coating without dripping or migration away from the target area. Additionally, such agents can provide a favorable feel and aesthetic to the lips during application and spreading of the lip color.

In an example, many classes of non-associative and associative thickeners can be used, provided they are selected to avoid an incompatibility with the chosen resin chemistry. In an example, hydrophilic gums and clays/silicates can be used. The specific type and level of structuring agent can be selected based on the desired level of performance for stability, application, and feel benefits of the composition, while minimizing adverse effects on durability of the applied film. Combinations of structuring agents may be used to achieve the aforementioned benefits.

In an example, the thickener is a hectorite. In an example, the thickener is disteardimonium hectorite or propylene carbonate.

Optionally, thickening/structuring agents may also be used in the separating top coat composition to improve application, feel, and/or long-wearing properties of the separating top coat composition. In an example, the total level of structuring agents can be about 0.05% to about 35% by weight of the total composition.

Top Coat Composition. The top coat composition includes components that are incompatible with the base coat composition and can be applied over the base coat composition upon application. The top coat composition can include one or more lipid-like materials that are insoluble with the base coat composition.

Following application of the base coat composition, the top coat composition is applied to impart lubricity, emollient feel, and shiny/glossy look to the surface of the pigmented coating.

The top coat composition can include a broad range of silicone-based, hydrocarbon-based or materials. The top coat composition is chosen so as to not act as a makeup remover on the base coat.

The top coat composition can have a low viscosity and lower surface energy relative to the pigmented, film-forming base coat composition. This can allow for application of the top coat composition to wet and spread across the base coat composition surface as desired.

The top coat can be oil or wax based. The top coat can include Helianthus annus, sunflower seed oil, polybutene, stearyl behenate, or ozokerite, among other components and/or emollients. The top coat composition components can include silicone fluids, fluorinated fluids, hydrocarbon fluids, both straight chain and branched, ester and ether fluids with low surface tension. The top coat can include a breakage of about 0.5 N to about 1.0 N.

To achieve improved a desired shine or gloss, the top coat composition can include one or more components with a Refractive Index (R.I.) greater than about 1.40, or greater than about 1.45. If the top coat composition includes a plurality of such materials, then at least 10% by weight, or 100% by weight, of the top coat composition can include materials with a R.I. greater than about 1.40.

In order to maintain sufficient insolubility/incompatibility between the base coat composition and the top coat composition, the difference in solubility parameter between the top coat composition and the base coat composition can be greater than about 1 (cal/cm³)^{1/2}.

Other Components. Additional optional ingredients such as flavors, fragrances, and skin-active or protecting agents, such as vitamins, sunscreens, NAG, Niacinamide, natural extracts, or others, may be included in the base coat composition and/or the top coat composition to enhance the experience or healthy positioning. These optional ingredients may be included in either the top coat composition or the base coat composition.

Applicator. In an example, the lip color can be provided as a dip-in tube with applicator wand, or a positive-displacement pen. A structured liquid, semi-solid, or paste formulation can be used for a positive displacement lipstick bullet. Combinations of these delivery mechanisms can be used. In some cases, similar delivery mechanisms can be used for the base coat composition and the top coat composition. In some cases, different delivery mechanisms can be used for the base coat composition and the top coat composition. Other delivery mechanisms are also envisioned.

### Method of Making

The lip color composition discussed herein can be made through a variety of methodologies. Discussed here is an example method of producing a long wear lip color having a long-wear base coat composition. The lip color can have a wear time of about 24 hours or more. The lip color can have a clean formulation.

In an example, a base coat composition can include a trimethylsiloxysilicate and polyphenylsilsesquioxane resin-based long wear lip color. The base coat composition can be made by a process of premixing the trimethylsiloxysilicate resin with a solvent and adding a plasticizer to make a preliminary mixture; heating the preliminary mixture above the glass transition temperature for polypheylsilsesquioxane to produce a heated mixture; suspending the polyphenylsilsesquioxane resin in the heated mixture and softening the heated mixture to produce a combined mixture; letting the combined mixture stand for a predetermined time to thicken and stabilize to produce an base coat composition; and combining the base coat composition with a thickener and optionally one or more pigments to produce the formulation.

Premixing the trimethylsiloxysilicate resin with a solvent and adding a plasticizer to make a preliminary mixture can include dispersing the resin and the plasticizer in a solvent such as isododecane. The plasticizer can be dimethicone. The plasticizer can have a D4/D5/D6 of less than 0.1%. The plasticizer can be about 13.00 wt % to about 15.00 wt % of the formulation. The plasticizer can have a minimum viscosity of about 900,000. Optionally, pigment can be mixed in at this time. The pigments can include coated pigments.

Heating the preliminary mixture can be done above the glass transition temperature of polypheylsilsequioxane, which is about 55 degrees Celsius. This can allow for efficient mixing of the two resins in the formulation, overall better tack, feel, and dryness. Suspending the polyphenylsilsesquioxane resin in the heated mixture and softening the heated mixture to produce a combined mixture can be done at a ratio of the polyphenylsilsesquioxane resin to the triethoxycaprylylsilane resin of about 5:1 to about 7:1. Addition of the polyphenylsilsesquioxane can provide structure to the formulation.

Letting the combined mixture stand for a predetermined time to thicken and stabilize to produce the base coat composition can allow for an appropriate viscosity and tack. The predetermined time can be about 45 minutes to about 90 minutes. Once made, the formulation can have a minimum viscosity of at least 3,500. The formulation can have a cyclic content of less than about 0.1% by weight. The formulation can include a water resistance of less than about 2. The formulation can include a tack of about 200 g to about 350 g. The formulation can include a moisture content of less than about 3.5. The formulation can include a long wear property of at least about 24 hours.

### Lip Color Examples

Various examples of the present disclosure can be better understood by reference to the following Examples which are offered by way of illustration. The present disclosure is not limited to the Examples given herein.

A series of experiments and tests were conducted on several base coat formulations of long-wear lip color, including clean experiments, wear experiments, and texture analysis. The formulations were both pre-tested for qualitative analysis and quantitively tested. These tests and results are summarized in Examples 1-3 below. Additionally, a series of experiments and tests were conducted on several top coat formulations, summarized in Example 4 below.

### Example 1. Plasticizers

Clean experiments were performed to determine whether the plasticizer in the formulation could be a green, clean, option for the base coat composition. The clean experiments included testing for formulation transfer and tack, flexibility, application, and drying time. Additionally, quantitative analysis for transferability, tack, drying time, stability, and coverage were performed.

Each sample contained the same formulation, with the plasticizer differing. The following samples, with differing plasticizers, were tested to compare, as shown in Table 1.

TABLE 1
Clean Testing Samples

| | Sample | Plasticizer | Viscosity | |
|---|---|---|---|---|
| Dimethicone | A | Dimethicone, 10-21% by weight | 100,000 | cPs |
| Screening | B | Dimethicone, 10-21% by weight | 300,000 | cPs |
| | C | Dimethicone, 10-21% by weight | 600,000 | cPs |
| | D1 | Dimethicone 1 Mcst, 10-15% by weight | 1,000,000 | cPs |
| | D2 | Dimethicone 1 Mcst, 15-21% by weight | 1,000,000 | cPs |
| Other | E | C26-28 Alkyl Methicone | Solid | |
| technologies | F | Aminopropyl Dimethicone blend | 40,000 | cPs |
| | G | Belsil ® 3096 (Dimethiconol blend) | 6,000 | mPa/sec |

| | | | |
|---|---|---|---|
| Associations | H | 50/50 blend of Belsil ® DM 3096 and | 6,000 mPa/sec |
| | | Dimethicone 1 Mcst, 10-15% by weight | (Belsil ® DM 3096) |
| | | (Belsil ® includes a blend of low- | |
| | | viscosity dimethicones and a high- | 1,000,000 cPs |
| | | molecular dimethiconol) | (Dimethicone 1 Mcst) |
| Controls | Control 1 | Internal non-clean composition | — |
| | Control 2 | Competitor composition | — |

Each of the dimethicone samples A, B, C, D1, and D2 was prepared in lab with dimethicone from Wacker Chemie AG (Munich, Germany) or Shin Etsu or Dow Corning at varying amounts by weight. Sample E was received from Dow Corning. Sample F was received from Shin Etsu. Sample G was received from Wacker Chemie AG (Munich, Germany). Sample H was blended in lab, with Belsil DM 3096 and dimethicone from Wacker Chemie AG (Munich, Germany) and Shin Etsu.

The samples had specific viscosities, with Sample D1 (Dimethicone 1Mcst, 10-15% by weight) and Sample D2 (Dimethicone 1 Msct 15-21% by weight) being the best viscosity, followed by Sample C (Dimethicone 10-21% by weight) and Sample H (Belsil® DM 3096+Dimethicone 1Mcst). As expected, the higher the amount of plasticizer, the higher the viscosity. However, it is desired for the viscosity of the sample to be balanced with flexibility, tack, and transfer, while maintaining an overall clean formulation with a lesser amount of cyclic content compared to the controls.

A series of qualitative tests were performed on the samples for flexibility, tack, and transfer. Transfer and tack were tested with a half/half application of lip color to lips, which was allowed to dry for five minutes. Cling paper was then applied to the lip color so that transfer of the lip color could be observed. Flexibility was tested with application of the lip color to a latex glove. The applied lip color was allowed to dry for 24 hours. The latex glove was then stretched to allow observation of the lip color flexibility. Application testing was done against a standard lip color. This allowed observation of viscosity, feel, look and comfort, compared to the standard lip color. Drying Time testing was done compared to standard lip color.

The results of the qualitative flexibility, tack, and transfer testing are summarized below in Table 2:

TABLE 2
Clean Testing Qualitative Results

| | Sample ID | | | | | | | | | Controls | |
|---|---|---|---|---|---|---|---|---|---|---|---|
| | A | B | C | D1 | D2 | E | F | G | H | 1 | 2 |
| Flexibility | Bad | Bad | Bad | Better | Good | Bad | Good | Good | Good | Good | Unknown |
| Tack | Good | Good | Good | Good | Bad | Good | Good | Good | Good | Good | Unknown |
| Transfer | Good | Good | Good | Good | Bad | Poor | Good | Good | Good | Good | Unknown |

Overall, Samples D1 (Dimethicone 1 Mcst, 15-20% by weight) and D2 (Dimethicone 1 Mcst, 10-15% by weight), Samples F (aminopropyl dimethicone blend) and G (dimethiconol blend), and Sample H (Blend of dimethiconol and dimethicone blend+dimethicone 1Mcst) performed best, with good results on all categories of flexibility, tack, and transfer, similar to in house Control 1. In Sample D, the flexibility of the sample improved with an increased percentage of dimethicone.

After these pre-tests, samples of interest were quantitively tested for transfer resistance, tack, drying time, stability, and coverage. These samples D1, F, G, and H, along with the Controls were further tested for this quantitative information. The samples were tested for transferability with a Crockmeter (Neurtek, Gipuzkoa, Spain) to determine the amount of color transferred from the samples to other surfaces by rubbing, in addition to being analyzed for tack and trying time with a Texture Analyzer, in addition to being tested for stability (qualitative) and coverage (by percent). The results are summarized in Table 3 below:

TABLE 3
Analysis of plasticizer samples

| | | Sample ID | | | | Controls | |
|---|---|---|---|---|---|---|---|
| | | D1 | F | G | H | 1 | 2 |
| Crockmeter | Transfer | 3.5 to 4.8 | 3.64 | 4.85 | 3.69 | 3.1 to 3.53 | 2.75 |
| | Resistance | | | | | | |
| Texture | Initial | 45.69 | 35.79 | — | 21.52 | 82.82 | 87.96 |
| Analyzer | Max | 255.11 | 220.58 | — | 251.07 | 208.135 | 236.35 |
| | Final | 104.83 | 170.24 | — | 307.34 | 109.75 | 93.38 |

| | | | | | | |
|---|---|---|---|---|---|---|
| Stability | Poor | — | — | Bad | Acceptable | Unknown |
| Coverage | 87% | — | — | — | 99% | 85% |

Here, Samples D1, F, and H, performed well in transfer resistance analysis with a Crockmeter value of between 3.5 and 4.8. Sample Samples C and H were further tested for stability compared to the Controls 1 and 2.

Sample D1, in addition to the Controls, were analyzed via a laboratory panel with consumers. In consumer testing, Sample D1 gave a similar 24-hour performance as Control 1, and better than Control 2. The Sample D1 had the best performance for overall liking in this test. Control 1 was liked the least by consumers, while Control 2 was middling.

Overall, for the clean experiments, a plasticizer with dimethicone in an amount of about 10% to 15% (Sample D1) provided flexibility as well as similar tack profiles and Crockmeter profiles as controls. This Sample D1 also fulfilled the desired clean formulation goals with a cyclic content of less than about 0.1%, while maintaining a good viscosity, tack, drying time, and consumer analysis.

### Example 2. Resins

Clean experiments were performed to determine whether the primary resin in the base coat composition formulation could be a green, clean, option. The clean experiments included testing for formulation transfer and tack, flexibility, application, and drying time. Additionally, quantitative analysis for transferability, tack, drying time, stability, and coverage were performed.

After conducting clean experiments with various plasticizers, a variety of wear experiments were conducted with differing resins. The various resins used in these wear experiments are summarized below in Table 4:

TABLE 4
Resin testing samples.

| Sample | Resin | % Resin | Resin Type |
|---|---|---|---|
| J | Trimethylsiloxysilicate in | 75% | Hard MQ |
| | Isododecane | | |
| K | Polymethylsilsesquixane | 100% | Hard MT |
| L | Trimethylsiloxysilicate (and) | 50% + 50% | Hard MQ + |
| | Polypropylsilsesquioxane | | Hard MT |
| M | Trimethylsiloxysilicate | 100% | Hard MQ |
| N | Trimethylsiloxysilicate | 100% | Soft MQ |
| O | Trimethylsiloxysilicate | 100% | Soft MQ |
| Control 1 | Internal non-clean composition | — | — |
| Control 2 | Competitor compositoin | — | — |

The resins were sources from a variety of vendors. Sample J was received from Shin Etsu. Sample K was received from Wacker Chemie AG (Munich, Germany). Samples L and M were received from Dow Chemical (Midland, MI). Samples N and O were received from Siltech Corp. (East York, Canada). These samples were mixed with the plasticizer Sample D1 (Dimethicone 1 Mcst, 10-15% by weight) in Example 1 for testing.

These samples were tested using pre-testing methods described above in Example 1, the results of which are shown in Table 5 below:

TABLE 5
Qualitative results for resin testing.

| | Sample ID | | | | | | Controls | |
|---|---|---|---|---|---|---|---|---|
| | J | K | L | M | N | O | 1 | 2 |
| Flexibility | Good | Poor | Bad | Bad | Good | Good | Good | Unknown |
| Tack | Good | Good | Good | Good | Bad | Bad | Good | Unknown |
| Transfer | Good | Good | Good | Good | Bad | Bad | Good | Unknown |

Here, Sample J (hard MQ resin trimethylsiloxysilicate in solvent isododecane), a Hard MQ resin in solvent, performed the best, giving good flexibility, tack, and transfer based on pre-testing. This resin was further tested for transfer resistance, tack, drying time, stability, and coverage using the same methods discussed with reference to Example 1 above. These results are summarized below in Table 6:

TABLE 6
Quantitative results for resin testing.

| | | Sample ID | Controls | |
|---|---|---|---|---|
| | | J | 1 | 2 |
| Crockmeter | Transfer Resistance | 3.5 to 4.28 | 3.1 to 3.53 | 2.75 |
| Texture | Initial | 45.69 | 82.82 | 87.96 |
| Analyzer | Max | 255.11 | 208.135 | 236.35 |
| | Final | 104.83 | 109.75 | 93.38 |

| | | | |
|---|---|---|---|
| Stability | Poor | Acceptable | Unknown |
| Coverage | 87% | 99% | 85% |

As can be seen by the transfer resistance results, Sample J had good wear results, indicating that a hard MQ resin in solvent had the best results. Subsequently, discussed in Example 3 below, secondary resins were tested with this primary resin to determine a beneficial overall formulation with good stability, coverage, viscosity, and application, while maintaining a green formulation with cyclic content of less than about 0.100.

### Example 3. Resin Mixes

After testing individual resins, clean experiments were performed to determine the best resin combinations for a green, clean, option in the base coat composition, by combining secondary resins with the 7500% hard MQ resin+25 solvent sample from Example 2. The clean experiments included testing for formulation transfer and tack, flexibility, application, and drying time. Additionally, quantitative analysis for transferability, tack, drying time, stability, and coverage were performed.

The various resins used in these wear experiments are summarized below in Table 7:

TABLE 7
Secondary resin testing samples.

| | | Ratio of Secondary |
|---|---|---|
| Sample | Resin | Resin to Main Resin |
| Q | Polyphenylsilsesquixane | 4:1 to 7:1 |
| R | C30-45 Alkyldimethylsilyl | 4:1 to 7:1 |
| | Polypropylsilsesquioxane | |
| S | Butyl Acrylate/Hydroxypropyl | 4:1 to 7:1 |
| | Dimethicone Acrylate Coresin | |
| T | Trimethylsiloxysilicate (60/40 in IDD) | 1:1 |
| U | Trimethylsiloxysilicate (100%) | 13:1 |
| Control 1 | Internal non-clean plasticizer | — |
| Control 2 | Competitor plasticizer | — |

Samples Q and U were received from Wacker Chemie AG (Munich, Germany). Sample R was received from Dow Chemical (Midland, MI). Sample S was received from Grant Industries Inc. (Elmwood Park, NJ). Sample T was received from Shin-Etsu Chemical (Tokyo, Japan). These samples were mixed with the plasticizer Sample D1 (Dimethicone 1 Mcst, 10-15% by weight) in Example 1 and Sample J (75% trimethylsiloxysilicate in isododecane) from Example 2 for testing.

These samples were tested using pre-testing methods described above in Example 1, the results of which are shown in Table 8 below:

TABLE 8
Qualitative results for secondary resin testing.

| | Sample ID | | | | | Controls | |
|---|---|---|---|---|---|---|---|
| | Q | R | S | T | U | 1 | 2 |
| Flexibility | Good | Bad | Acceptable | Good | Good | Good | Unknown |
| Tack | Good | Good | Good | Good | Good | Good | Unknown |
| Transfer | Good | Good | Good | Poor | Good | Good | Unknown |

Here, Q, S, and U performed the well, giving good flexibility, tack, and transfer based on pre-testing. These samples were further tested for transfer resistance, tack, drying time, stability, and coverage using the same methods discussed with reference to Example 1 above. These results are summarized below in Table 9:

TABLE 9
Quantitative results for secondary resin testing.

| | | Sample ID | | | Controls | |
|---|---|---|---|---|---|---|
| | | Q | S | U | 1 | 2 |
| Crockmeter | Transfer | 2.3 to 2.4 | 4.4 | 2.5 | 3.1 to 3.53 | 2.75 |
| | Resistance | | | | | |
| Texture | Initial | 66.53 | 75 | 30.95 | 82.82 | 87.96 |
| Analyzer | Max | 283.85 | 245.24 | 241.56 | 208.135 | 236.35 |
| | Final | 166.33 | 121.74 | 131.91 | 109.75 | 93.38 |

| | | | | | |
|---|---|---|---|---|---|
| Stability | Approved | Failed | Bad | Acceptable | Unknown |
| Coverage | 98% | — | 86% | 99% | 85% |

As can be seen by the transfer resistance, tack and drying time (texture analyzer) results, Sample Q (polyphenylsilsesquixane) performed the best of these. This had better coverage and stability overall. Sample U (Trimethylsiloxysilicate (100%)) also performed well.

These two samples were tested in a laboratory panel, sensory test, and for clinical wear. The laboratory panel testing assigned the samples a grade from 0 (bad) to 5 (good) based on standard ClearCast procedures. The sensory testing qualitatively analyzed the long wear 24-hour performance of the formulations. The clinical long wear testing assigned a score less than or equal to 3.00 based on 24 hours long wear performance.

These results are summarized in Table 10 below:

TABLE 10
Consumer testing results for secondary resin formulations.

| | | Sample ID | | Controls | |
|---|---|---|---|---|---|
| | | Q | U | 1 | 2 |
| Laboratory Panel | | 3.23 to 3.45 | 3.53 | 2.57 | 3.00 |
| Sensory | Lip | Better lip | Better lip | Lower lip | Lower lip |
| Testing | coverage | coverage | coverage | coverage after | coverage after |
| | | compared to | compared to | 24 hours | 24 hours |
| | | controls | controls | | |
| | Residuals | Better pay off | Better pay off | Lower pay off | Lower pay off |
| | | residual | residual | residual after 24 | residual after 24 |
| | | compared to | compared to | hours | hours |
| | | controls | controls | | |
| | Tack | Tackier than | Tackier than | Low tack | Low tack |
| | | controls | controls | | |

| Clinical Wear | 2.48 | 2.52 | 2.40 | 2.23 |
|---|---|---|---|---|
| | (at 60%) | (at 60%) | (at 70%) | (at 80%) |

The formulation with Sample D1 (Dimethicone 1 Mcst, 10-15% by weight) in Example 1, Sample J (75% trimethylsiloxysilicate in isododecane) from Example 2, and Sample Q (polyphenylsilsesquixane) from Example 3 performed the best overall.

### Example 4. Top Coat Compositions

Additionally, clean experiments were performed to investigate top coat compositions to work with the base coat composition. Top coat components were investigated to look at top coat formulations having low compatibility with the base coat composition, so as to prevent the top coat from acting as a make-up remover and decreasing overall wear of the base coat composition. A non-exhaustive list of samples top coat component tested and screened for compatibility are summarized below:

TABLE 11
Top coat composition component testing.

| ID | Component(s) | Source | Compatibility |
|---|---|---|---|
| AA | Bis-digylchey polyacyladipate - 2 | IOI Oleo GmbH | 1.764 |
| | | Hamburg, | |
| | | Germany | |
| BB | Punica Granatum Sterols | Active Concepts | 1.84 |
| | | LLC Lincoln, NC | |
| CC | Butyl Acrylate/Hydroxypropyl Dimethicone Acrylates Coresin | Grant Industries, | 2.175 |
| | | Inc. Elmwood | |
| | | Park, NJ | |
| DD | Dimethicone (6000 cst) | Dow Chemical | 3.277 |
| | | Midland, MI | |
| EE | Castor Oil/IPDI Coresin | ALZO | 3.627 |
| | | International Inc. | |
| | | Sayreville, NJ | |
| FF | Dimethicone (12500 cst) | Dow Chemical | 4.192 |
| | | Midland, MI | |
| GG | Helianthus Annuus (Sunflower) seed oil | Flora | 4.742 |
| | | Technologies | |
| | | Minnetonka, MN | |
| HH | Trioctyldodecyl Citrate | ALZO | 5.17 |
| | | International Inc. | |
| | | Sayreville, NJ | |
| II | Polyglyceryl-10 Pentaoleate | Barnet Products | 5.742 |
| | | Englewood Cliffs, | |
| | | NJ | |
| JJ | PPG-51/SMDI Coresin | Barnet Products | 5.826 |
| | | Englewood Cliffs, | |
| | | NJ | |
| KK | VP/Hexadecene Coresin | Givaudan Active | 5.841 |
| | | Beauty | |
| LL | Polybutene (235 cst) | INEOS Oligomers | 5.943 |
| | | USA LLC | |
| | | Alvin, TX | |
| MM | Tridecyl Trimellitate | Vantage Personal | 6.721 |
| | | Care | |
| | | Chicago, IL | |
| NN | Polyglyceryl-2 Diisostearate | ALZO | 6.741 |
| | | International Inc. | |
| | | Sayreville, NJ | |
| OO | Dipentaerythrityl Hexa C5-9 Acid Esters | Inolex | 6.953 |
| | | Philadelphia, PA | |
| PP | Phenylpropyldimethylsiloxysilicate | Momentive | 7.286 |
| | | Performance | |
| | | Materials | |
| | | Waterford, NY | |
| QQ | Ethylhexyl Hydroxystearate | Lubrizol | 7.55 |
| | | Wickliffe, OH | |
| RR | Di-PPG-3 Myristyl Ether Adipate AND Pentaerythrityl Tetra- | Croda Inc. | 8.212 |
| | di-t-butyl Hydroxyhydrocinnamate | Snaith, UK | |
| SS | Diisopropyl Dimer Dilinoleate | Lubrizol | 10.655 |
| | | Wickliffe, OH | |
| TT | Hydrogenated Polyisobutene | Vantage Personal | 19.096 |
| | | Care | |
| | | Chicago, IL | |

Of the samples summarized in Table 11 and tested for compatibility, Samples AA (Bis-digylchey polyacyladipate—2), BB (Punica Granatum Sterols), GG (Helianthus Annuus (Sunflower) seed oil), HH (Trioctyldodecyl Citrate), and LL (Polybutene (235 cst)) showed potential for good compatibility with the base coat composition. These samples had relatively good feel and balance wear benefits.

These samples were used as emollients in sample top coats, which were qualitatively and quantitatively tested as described with reference to Example 1 above. These results are summarized below in Table 12:

TABLE 12
Testing of top coat compositions.

| | | | Feel (Tack, Glide, and | Transfer |
|---|---|---|---|---|
| ID | Main Emollient | Ratio | Comfort) | (Crockmeter) |
| AA | Bis-Diglyceryl Polyacyladipate-2 | 1 | Bad | NA |
| BB | Punica Granatum Sterols | 1 | Bad | NA |
| GB | Helianthus Annus seed oil + | 0.8 to 1.2 | Slightly heavier than | 6.65 |
| | Punica Granatum Sterols | | control | |
| GH1 | Helianthus Annus seed oil + | 0.8 to 1.2 | Similar to control | 7.05 |
| | Trioctyldodecyl Citrate | | | |
| GL1 | Helianthus Annus seed oil + | 0.8 to 1.2 | Similar to control | 8.89 |
| | Polybutene | | | |
| GL2 | Helianthus Annus seed oil + | 1.9 to 2.3 | Closest to control | 5.96 |
| | Polybutene | | | |
| GH2 | Helianthus Annus seed oil + | 1.9 to 2.4 | Similar to control | 6.37 |
| | Trioctyldodecyl Citrate | | | |
| | Top Coat Control | — | — | 7.35-8.71 |

Overall, the top coat mixture containing both Helianthus Annus seed oil and Polybutene (Sample GL1) at a ratio of about 0.8:1 to about 1.2:1 was the closest to the control top coat.

### Example 5. Clinical Testing

Several sample lipsticks of both a base coat and a top coat were tested in clinical evaluation. The samples were tested for the efficacy of the formulation for a 24-hour period. The formulation was applied in a single application on the lips of female subjects by a technician. Thirty three subject were tested ages 19 to 52, with a median age of 30. The formulation was then digitally photographed in cross-polarized light by an expert. The photographs were taken at 0 and 24 hours.

Clinical evaluation was conducted to evaluate the presence and the homogeneity of the combined base coat and a top coat formulation. Conclusions about long term wear were recorded on a scheduled evaluation at the median time and at the full 24-hour time. These evaluations are carried out by one expert using a scale of photographs with 5 grades (with half grades allowed), as summarized in Table 13:

TABLE 13
Clinical testing evaluation scale.

| | | Comparison |
|---|---|---|
| | | to Median |
| No. | Description | Time |
| 4 | Even characteristics like after the application | 100% |
| 3 | Characteristics very close to those of the application | 75% |
| 2 | Characteristics quite close to that of the application | 50% |
| 1 | Characteristics not very close to that of the | 25% |
| | application | |
| 0 | Characteristics not at all those of the application | 0 |

At the 24 hour time mark, if the presence of the formulation was scored at a value of 0.5 or more, then the homogeneity, long lasting wearing, and global wear of the formulation was also evaluated and scored. To determine if the product did not move until the 24 hour time, the median had to be greater than or equal to 2.5 on a scale of 5.

These evaluations were run on the following samples:

TABLE 14
Clinical testing samples.

| Sample | Base Coat | Top Coat |
|---|---|---|
| Clinical A | A | A |
| Clinical B | B | B |
| Clinical C | C | C |
| Clinical D | D | None |

Clinical A was a legacy base coat and top coat sample. Clinical B was a commercially available two-step lip color for comparison. Clinical C was the inventive sample base coat with a top coat. Clinical D was the inventive sample including the base coat composition including silicone-based resins include at least an MQ resin and an MT resin, with an MT:MQ ratio of about 3:1 to about 7:1, a treated pigment, and a thickener.

The median values for each of presence, homogeneity, long lasting wearing, and global wear, with the median evaluations shown in Table 15:

TABLE 15
Clinical testing median results.

| | Presence of | Homogeneity | Long lasting | Global wear at |
|---|---|---|---|---|
| | product | of product | wearing | 24 hours |
| Clinical A | 3.0 | 3.0 | 3.0 | yes |
| Clinical B | 2.5 | 2.0 | 2.0 | N/A |
| Clinical C | 3.5 | 3.0 | 3.3 | yes |
| Clinical D | 3.5 | 3.0 | 3.3 | yes |

As can be seem in Table 15, the Clinical D, which included the inventive base coat without a top coat, outperformed even Clinical A and Clinical B, both of which had a top coat. Clinical D performed commensurate with the Clinical C sample.

The terms and expressions that have been employed are used as terms of description and not of limitation, and there is no intention in the use of such terms and expressions of excluding any equivalents of the features shown and described or portions thereof, but it is recognized that various modifications are possible within the scope of the examples of the present disclosure. Thus, it should be understood that although the present disclosure has been specifically disclosed by specific examples and optional features, modification and variation of the concepts herein disclosed may be resorted to by those of ordinary skill in the art, and that such modifications and variations are considered to be within the scope of examples of the present disclosure.

### Additional Examples

The following exemplary examples are provided, the numbering of which is not to be construed as designating levels of importance:

Example 1 is a long-wear lip color formulation comprising: a base coat composition having a cyclic content of less than 0.1% by weight, the base composition comprising: a first resin comprising polyphenylsilsesquioxane; a second resin comprising trimethylsiloxysilicate, wherein the ratio of trimethylsiloxysilicate to polyphenylsilsesquioxane is about 4:1 to about 7:1 in the base coat composition; a plasticizer comprising dimethicone; a thickener; at least one pigment; and a solvent, wherein the first resin, the second resin, the thickener, the plasticizer, and the at least one pigment are in the solvent; and a top coat composition, wherein, once the base coat composition and the top coat composition are applied sequentially, the long-wear lip color formulation having a wear time of about 24 hours or more.

In Example 2, the subject matter of Example 1 optionally includes wherein the base coat composition has a viscosity of at about 3,500 cPs to about 110,000 cPs.

In Example 3, the subject matter of Example 2 optionally includes wherein the base coat composition has a viscosity of about 5,000 cPs to about 20,000 cPs.

In Example 4, the subject matter of any one or more of Examples 1-3 optionally include wherein the base coat composition has a viscosity of at least about 5,000 cPs.

In Example 5, the subject matter of Example 4 optionally includes wherein the base coat composition has a viscosity of at least about 10,000 cPs.

In Example 6, the subject matter of Example 5 optionally includes wherein the base coat composition has a viscosity of at least about 25,000 cPs.

In Example 7, the subject matter of any one or more of Examples 1-6 optionally include wherein the plasticizer has a viscosity of at least about 900,000 cPs.

In Example 8, the subject matter of Example 7 optionally includes wherein the plasticizer has a viscosity of about 900,000 cp to about 1,000,000 cPs.

In Example 9, the subject matter of any one or more of Examples 1-8 optionally include the ratio of trimethylsiloxysilicate to polyphenylsilsesquioxane is about 5:1 in the base coat composition.

In Example 10, the subject matter of any one or more of Examples 1-9 optionally include wherein the first resin comprises about 3% to about 10% by weight of the base coat compositions.

In Example 11, the subject matter of any one or more of Examples 1-10 optionally include wherein the second resin comprises about 10% to about 60% by weight of the base coat composition.

In Example 12, the subject matter of any one or more of Examples 1-11 optionally include wherein the thickener comprises disteardimonium hectorite or propylene carbonate.

In Example 13, the subject matter of any one or more of Examples 1-12 optionally include wherein the thickener comprises a hectorite.

In Example 14, the subject matter of any one or more of Examples 1-13 optionally include wherein the plasticizer comprises about 20% or less by weight of the formulation.

In Example 15, the subject matter of any one or more of Examples 1-14 optionally include wherein the at least one pigment comprises a treated pigment.

In Example 16, the subject matter of Example 16 optionally includes wherein the at least one pigment is triethoxycaprylylsilane treated.

In Example 17, the subject matter of any one or more of Examples 1-16 optionally include wherein the solvent comprises isododecane.

In Example 18, the subject matter of any one or more of Examples 1-17 optionally include wherein the second resin, the thickener, the plasticizer, and the at least one pigment are dispersed in the solvent.

In Example 19, the subject matter of any one or more of Examples 1-18 optionally include wherein the first resin is suspended in the solvent.

In Example 20, the subject matter of any one or more of Examples 1-19 optionally include wherein the top coat composition comprises an oil-based top coat composition.

In Example 21, the subject matter of Example 20 optionally includes wherein the top coat composition comprises Helianthus annus, sunflower seed oil, polybutene, stearyl behenate, or ozokerite.

In Example 22, the subject matter of any one or more of Examples 19-21 optionally include wherein the top coat composition comprises sunflower seed oil.

In Example 23, the subject matter of any one or more of Examples 1-22 optionally include wherein the long-wear lip color formulation has a wear time of about 24 hours or more according to consumer clinical screening.

In Example 24, the subject matter of any one or more of Examples 1-23 optionally include wherein the base coat composition with the top coat composition layered thereon has a wear time of about 24 hours or more according to consumer clinical screening.

Example 25 is a long-wear lip color composition comprising: a base coat composition comprising base coat components including a film-forming silicone-based resin, at least one pigment, a thickener, and a plasticizer, in a solution, wherein the silicone-based resins comprise at least an MQ resin and an MT resin, having a ratio of about 3:1 to about 8:1; and a top coat composition applied sequentially on the base coat composition to create a long-wearing film on an integument, wherein the long-wearing film on an integument has a wear time of at least 24 hours.

In Example 26, the subject matter of Example 25 optionally includes wherein the at least one pigment is triethoxycaprylylsilane treated.

In Example 27, the subject matter of any one or more of Examples 1-26 optionally include wherein the silicone-based resins comprise at least polyphenylsilsesquioxane and trimethylsiloxysilicate.

In Example 28, the subject matter of any one or more of Examples 1-27 optionally include wherein the MQ resin, and the MT resin have a ratio of about 4:1 to about 7:1.

In Example 29, the subject matter of any one or more of Examples 1-28 optionally include wherein the silicone-based resins comprise about 10% up to about 65% by weight of the base coat composition.

In Example 30, the subject matter of Example 29 optionally includes wherein the silicone-based resins comprise about 10% up to about 50% by weight of the base coat composition.

In Example 31, the subject matter of any one or more of Examples 1-30 optionally include wherein the base coat composition further comprises a thickener.

In Example 32, the subject matter of Example 31 optionally includes wherein the thickener comprises a clay or silicate.

In Example 33, the subject matter of any one or more of Examples 30-32 optionally include wherein the thickener comprises about 0.05% to about 40% by weight of the base coat composition.

In Example 34, the subject matter of any one or more of Examples 1-33 optionally include wherein the pigment comprises iron oxides, titanium dioxide, FD&C dyes, D&C dyes, D&C lakes, micas, pearlescent powders, and combinations thereof.

In Example 35, the subject matter of any one or more of Examples 1-34 optionally include wherein the plasticizer comprises about 20% or less by weight of the composition.

In Example 36, the subject matter of any one or more of Examples 1-35 optionally include wherein the plasticizer comprises dimethicone.

In Example 37, the subject matter of Example 35 optionally includes wherein the plasticizer comprises a viscosity of at least about 900,000 cPs.

In Example 38, the subject matter of any one or more of Examples 35-37 optionally include wherein the plasticizer comprises a D4/D5/D6 of less than about 0.1%.

In Example 39, the subject matter of any one or more of Examples 1-38 optionally include wherein the top coat composition comprises Helianthus annus, sunflower seed oil, polybutene, stearyl behenate, or ozokerite.

In Example 40, the subject matter of any one or more of Examples 1-39 optionally include wherein the top coat composition has a breakage of about 0.5 N to about 1 N.

In Example 41, the subject matter of any one or more of Examples 1-40 optionally include flavors, fragrances, skin-active agent, skin protecting agents, and combinations thereof.

Example 42 is a formulation including a trimethylsiloxysilicate and polyphenylsilsesquioxane resin-based long wear lip color, wherein the formulation is made by a process of: premixing the trimethylsiloxysilicate resin with a solvent and adding a plasticizer to make a preliminary mixture; heating the preliminary mixture above the glass transition temperature for polypheylsilsesquioxane to produce a heated mixture; suspending the polyphenylsilsesquioxane resin in the heated mixture and softening the heated mixture to produce a combined mixture; letting the combined mixture stand for a predetermined time to thicken and stabilize to produce an base coat composition; and combining the base coat composition with a thickener and optionally one or more pigments to produce the formulation, wherein the lip color has a wear time of about 23 hours or more.

In Example 43, the subject matter of Example 42 optionally includes wherein the glass transition temperature comprises 55 °C.

In Example 44, the subject matter of any one or more of Examples 42-43 optionally include the formulation has a minimum viscosity of at least 3,500 cPs.

In Example 45, the subject matter of any one or more of Examples 42-44 optionally include wherein the formulation has a cyclic content of about 0.1% or less by weight.

In Example 46, the subject matter of any one or more of Examples 42-45 optionally include wherein the plasticizer comprises dimethicone.

In Example 47, the subject matter of any one or more of Examples 42-46 optionally include wherein the plasticizer comprises about 10.00 wt. % to about 20.00 wt. % of the formulation.

In Example 48, the subject matter of any one or more of Examples 42-47 optionally include wherein the plasticizer has a minimum viscosity of about 900,000 cPs.

In Example 49, the subject matter of any one or more of Examples 42-48 optionally include wherein the ratio of the polyphenylsilsesquioxane resin to the triethoxycaprylylsilane resin comprises about 4:1 to about 7:1.

In Example 50, the subject matter of any one or more of Examples 42-49 optionally include mixing pigments into the preliminary mixture, the pigments comprising coated pigments.

In Example 51, the subject matter of any one or more of Examples 42-50 optionally include wherein letting the combined mixture stand comprises letting the mixture reach a thickness of about 7,000 cPs to about 12,000 cPs.

In Example 52, the subject matter of any one or more of Examples 42-51 optionally include wherein the formulation comprises a water resistance of less than about AE 2.5 with deionized water.

In Example 53, the subject matter of any one or more of Examples 42-52 optionally include wherein the formulation comprises a tack of about 60 g to about 275 g.

1. A long-wear lip color formulation comprising:
a base coat composition having a cyclic content of less than 0.1% by weight, the base composition comprising:
a first resin comprising polyphenylsilsesquioxane;
a second resin comprising trimethylsiloxysilicate, wherein a ratio of trimethylsiloxysilicate to polyphenylsilsesquioxane is between 4:1 and 7:1 by weight in the base coat composition;
a plasticizer comprising dimethicone;
a thickener;
at least one pigment; and
a solvent, wherein the first resin, the second resin, the thickener, the plasticizer, and the at least one pigment are in the solvent; and
a top coat composition,
wherein, when the base coat composition and the top coat composition are applied sequentially, the long-wear lip color formulation is capable of having a wear time of at least 24 hours.

2. The long-wear lip color formulation of claim 1, wherein the base coat composition has a viscosity between 3,500 cPs and 110,000 cPs.

3. The long-wear lip color formulation of claim 1, wherein the base coat composition has a viscosity greater than 7,500 cPs.

4. The long-wear lip color formulation of claim 3, wherein the base coat composition has a viscosity greater than 15,000 cPs.

5. The long-wear lip color formulation of claim 1, wherein the plasticizer has a viscosity of greater than 900,000 cPs.

6. The long-wear lip color formulation of claim 5, wherein the plasticizer has a viscosity between 900,000 cp and 1,000,000 cPs.

7. The long-wear lip color formulation of claim 1, the ratio of trimethylsiloxysilicate to polyphenylsilsesquioxane is 5:1 by weight in the base coat composition.

8. The long-wear lip color formulation of claim 1, wherein the first resin comprises between 3% and 10% by weight of the base coat compositions.

9. The long-wear lip color formulation of claim 1, wherein the second resin comprises between 10% and 60% by weight of the base coat composition.

10. The long-wear lip color formulation of claim 1, wherein the thickener comprises disteardimonium hectorite or propylene carbonate.

11. The long-wear lip color formulation of claim 1, wherein the thickener comprises a hectorite.

12. The long-wear lip color formulation of claim 1, wherein the plasticizer comprises less than 20% by weight of the long-wear lip color formulation.

13. The long-wear lip color formulation of claim 1, wherein the at least one pigment comprises a treated pigment.

14. The long-wear lip color formulation of claim 1, wherein the solvent comprises isododecane.

15. The long-wear lip color formulation of claim 1, wherein the second resin, the thickener, the plasticizer, and the at least one pigment are dispersed in the solvent.

16. The long-wear lip color formulation of claim 1, wherein the first resin is suspended in the solvent.

17. The long-wear lip color formulation of claim 1, wherein the top coat composition comprises an oil-based top coat composition.

18. The long-wear lip color formulation of claim 17, wherein the top coat composition comprises Helianthus annus, sunflower seed oil, polybutene, stearyl behenate, or ozokerite.

19. The long-wear lip color formulation of claim 1, wherein the long-wear lip color formulation has a wear time of at least 24 hours according to consumer clinical screening.

20. The long-wear lip color formulation of claim 1, wherein the base coat composition with the top coat composition layered thereon has a wear time of at least 24 hours according to consumer clinical screening.
